(19) United States
(12) Patent Application Publication
(10) Pub. No.: US 2008/0248508 A1
(43) Pub. Date: Oct. 9, 2008

(54) Methods of making a chitosan product having an ultra-low endotoxin concentration and the ultra-low endotoxin chitosan product derived therefrom and method of accurately determining inflammatory and anti-inflammatory cellular response to such materials

(76) Inventors: Shenda Baker, Upland, CA (US); William P. Wiesmann, Washington, DC (US)

Correspondence Address: BARTUNEK & BHATTACHARYYA, LTD., 10420 LITTLE PATUXENT PARKWAY, SUITE 405, COLUMBIA, MD, 21044-3533, US

(21) Appl. No.: 11/985,057

(22) Filed: Nov. 13, 2007

Related U.S. Application Data
(63) Continuation-in-part of application No. 11/657,382, filed on Jan. 24, 2007.
(60) Provisional application No. 60/858,576, filed on Nov. 13, 2006.
(60) Provisional application No. 60/838,790, filed on Aug. 17, 2006.

(51) Int. Cl. G01N 33/53; C08B 37/08

(52) U.S. Cl. 435/7.92; 536/20

(57) ABSTRACT

Chitosan is a natural product having wide range of applications in the food and cosmetic industries. Food and commodity grade chitosan are laden with pyrogens, such as endotoxins and proteins which limit its applicability in the biological and medical arenas, as minute amounts of endotoxins may induce adaptive and innate responses when contacted with mammalian tissue, pharmaceuticals and biomedical devices. Due to chitosan's ability to avidly bind endotoxin and other pyrogens, they are difficult to remove. The present invention is directed to methods for purifying chitosan from shells, food and commodity grade chitosan into ultra-pure, low endotoxin chitosan having biological and medical applicability. Additionally, the present invention is also directed to a method of determining the pyrogenicity of the ultra-pure low endotoxin chitosan.

### PRIORITY INFORMATION

This application obtains priority from Provisional Application 60/858,576, filed Nov. 13, 2006 and also is a Continuation-in-part of application Ser. No. 11/657,382, filed Jan. 24, 2007 which obtains priority from Provisional Application 60/838,790, filed Aug. 18, 2006.

### STATEMENT OF GOVERNMENT INTEREST

The United States Government shall have a nonexclusive, nontransferable, irrevocable, paid-up license to practice or have practiced for or on behalf of the United States the subject invention.

### BACKGROUND OF THE INVENTION

In order to coexist with bacteria, viruses, parasites and other pathogens, higher organisms have developed highly functionalized and specialized immune systems. A host organism must strike a balance between tolerating commensal flora and being overrun by that same flora or exogenous pathogens. Vertebrate animals, for example, have developed innate and adaptive immune recognition systems.

In mammals, particularly humans, the adaptive immune recognition system has long been considered to be the crusader in the fight against infectious agents. However, this system provides a narrowly selective lethal response to broad insults that is slow to respond to rapidly growing endogenous and exogenous microbes. More recently, studies show that the innate immune recognition system manipulates the effector class of the adaptive immune recognition system and forms the first line of host defense. The innate immune recognition is thus, an ancient but evolved system that detects and responds to immediate cellular level threats, controls the initiation of the adaptive immune recognition system (if applicable) and signals for rapid and local protection. The innate immune recognition system encompasses a network of pattern recognition receptors (PRR) that identify pathogen associated molecular patterns (PAMPs). The network of PRRs in the host, controls pathogens long before the adaptive system gets underway. See Medzhitov, “Toll-like Receptors and Innate Immunity,” Nature Reviews: Immunology, Vol. 1, p 135-145 (2001); and Sansonetti, “The Innate Signaling Of Dangers And The Dangers Of Innate Signaling,” Nature Publishing Group, Vol. 7, No. 12, p. 1237-1242 (2006).

A variety of types of receptors have been identified as PRR's in the innate immune system. The most common is the Toll-like receptor (TLR), which are essential to animal immunity. In mammals, these transmembrane receptors are expressed on the surface of dendritic cells, macrophages and epithelial cells and recognize specific PAMP's by direct binding to them. See id.

These TLRs are identified in Table 1.

TABLE 1
Ligands to known Toll-like Receptors

| TLR | Binds | Example |
|---|---|---|
| TLR1 | Fungi, dimerizes with TLR2 | |
| TLR2 | Peptidoglyans (PG's) of | Streptococci, Staphylococci |
| | Gram+ bacteria | |
| TLR3 | Double stranded DNA (dsDNA) | Viruses, particularly as part |
| | or ds RNA | of their infection cycle |
| TLR4 | Lipopolysaccharide (endotoxin) | Salmonella, E. coli O157:H7 |
| | of Gram− bacteria | |
| TLR5 | Flagellin of motile bacteria. | Listeria |
| | a conserved protein | |
| LTR6 | Heterodimer with TLR2 to | |
| | bind PG and certain lipids | |
| TLR7 | Single stranded RNA (SSRNA) | Viruses influenza, measles, |
| | | and mumps |
| TLR8 | SsRNA | |
| TLR9 | Unmethylated CpG of DNA | Anything non host as hosts |
| | | typically have methyl groups |
| | | attached, most bacterial DNA |
| TLR11 | (in mice) proteins from | Apicomplexa |
| | infectious protozoa | |

Upon binding their target, the TLR's initiate signaling pathways and the shared activation of MyD88 and NF-κβ, although the TLR's may also initiate specific pathways as well. The primary response to PAMPs is local inflammation induced by the expression of inflammatory cytokines such as IL-1β, TNF-α, IL-6, IL-8 and IL-12 and chemokines which attract white blood cells to the site. Mammalian TLRs can also induce multiple effector molecules such as nitric oxide synthase and antimicrobial peptides, which directly kill microbial pathogens. Of particular interest is the powerful role of TLR2 and TLR 4. These receptors respond primarily to bacteria to produce TNF-α and the interleukins IL-8 and IL-1β.

Other types of innate receptors such as C-type lectin receptors (CLR's) and nucleotide-binding oligomerization domain-like receptors (Nod-like receptors or NLR's) play less visible roles, but are also responsible for maintaining a healthy balance between the host and its flora. For example, the lack of NLR function in the bowel has been linked to Crohns's disease.

Endotoxins are complex amphiphilic lipopolysaccharides (LPS) having both polysaccharide and lipophilic components. They are composed of pieces of the lipopolysaccharide wall component of Gram-negative bacteria as shown in FIG. 1 and FIG. 2. Endotoxins, like bacteria, are ubiquitous and found in the air, on surfaces and in food and water. The biological activity of endotoxin is associated with the lipid component (Lipid A) while the immunogenicity is associated with the polysaccharide components of the LPS. As shown in FIG. 1, the surfactant behavior of endotoxin makes it soluble in a variety of solvents because the more favorable component can be exposed, resulting in micelles in aqueous solvents and inverse micelles in organic solvents.

Endotoxins are highly toxic to mammals, particularly humans. As such, they are an exquisitely sensitive activator of immune response in cells, primarily through TLR4 stimulating a variety of cytokines involved in inflammatory and wound healing response and initiating the body's acute response to bacterial infection. Endotoxin, which binds to TLR4, stimulates a very large TNF-α response while Gram-positive PG's such as lipoteichoic acid produce lower TNF-α but more IL-8. See Konrad et al., “Differences in innate immune responses upon stimulation with Gram-positive and Gram-negative bacteria,” J of Periodontal Research Vol. 41, p. 447-454 (2006).

However, upon sustained stimulation of TLR4 by endotoxin, a potentially fatal clinical condition called endotoxic shock can be produced. This endotoxemia is caused by the hyper-reaction of the innate immune system to a bacterial load. While a healthy response to bacteria is necessary, a delicate balance exists in all innate signaling between inflammation and apoptosis (cell death) and tolerance or regulation. Inflammation leads to destruction of the microbes and surrounding tissue, but lack of control or inadequate regulation can lead to severe sepsis or septic shock.

Endotoxin is notoriously difficult to remove from materials. The surfactant behavior of endotoxin makes it attractive to hydrophobic and hydrophilic moieties as well as to positively charged species. These properties make endotoxin very difficult to remove from both polar and non-polar materials. This same surfactant behavior makes endotoxin adhere to both hydrophilic and hydrophobic surfaces. It is not particularly soluble as a single molecule as it will from regular or inverse micelles depending on its environment. At present, techniques of endotoxin removal are highly limited by these physical properties.

As discussed above, inflammatory responses are indicative of pathogenic contamination in organisms. Pyrogens are a heterogenous group of compounds that induce immune responses that are also indicative of pathogenic contamination. In mammals, particularly humans, pyrogenic responses manifest in fever. Endotoxin is one of the most common pyrogens, and many methods of depyrogenation refer to removal of endotoxin. See Depyrogenation, Technical Report No. 7, Parenteral Drug Association, Inc. at 3. Table 1, above, lists other pyrogens (endogenous biological contaminants) that induce fever and inflammation throughout the TLR's.

Treatments to remove or destroy pathogens, particularly, endotoxin are referred to as methods of “depyrogenation”. For the depyrogenation of materials containing endotoxin, techniques involve oxidation by hydrogen peroxide, chemical alkylation and dry heat not less than 250 C for no less than 30 min (pharmaceutical industry standard), which presumably causes thermal degradation of the bonds. See id.

The detoxification of endotoxin by acid and base are methods that destroy the endotoxin by chemically modifying the bonds in the toxin. Although each bacterial strain responds somewhat differently, all methods involve processing at high temperatures for extended periods of time and warn that each method must be validated. Furthermore, the common assay for endotoxin, the limulus amoebate lysate assay (LAL) assay discussed below, is unpredictable for the pyrogenic properties of endotoxin in humans. Thus, a carefully instituted validation process must be used in order to determine the efficacy of the acid/base detoxification.

A number of methods exist for removing endotoxins from solutions. These include ultrafiltration, application of reverse osmosis, charge modified media, microporous membrane filtration, sintered activated carbon and ion exchange columns. The polycationic peptide, Polymixin B, is one of the most potent binders of endotoxin. It is used in columns to bind endotoxin and separate it from solution or to bind it and render it inactive.

All biologically derived materials begin in an environment where they are exposed to exogenous and endogenous microbes and flora. While the pure material itself may be entirely benign in a mammal or animal, it can cause serious and robust inflammatory responses if the material is not properly and completely purified.

Chitosan is a polycationic biopolymer that is a biocompatible natural material derived primarily from the shells of arthropods and from fungi. While it used in numerous commodity applications such as metal flocculation in water, feed stock additive, over the counter weight loss and filler, foaming and adsorbent, chitosan has potential application in the medical fields. In its purest form, chitosan may be utilized as internal hemostatic dressings, as a drug delivery agent, as tissue scaffolding and in multiple other health related products.

Chitosan carries a variety of contaminants, common to biologically derived materials, and thus must be carefully purified and its resultant stimulatory immune responses in humans and animals well understood before use in medically related applications. As a biopolymer, chitosan has various levels of bioburden in the available products as a result of the impurities in the source as well as impurities introduced in the processing. In particular, naturally derived chitosan contains variable amounts of endotoxin and residual proteins. Because of the biological source of chitosan from chitin and non-pyrogen-free processing, endotoxins from bacteria associated with the species and/or exogenous bacteria remain, albeit in small quantities, in the resultant polysaccharide. Chitosan is a polycationic biopolymer that is a biocompatible natural material derived primarily from the shells of arthropods and from fungi. While it used in numerous commodity applications such as metal flocculation in water, feed stock additive, over the counter weight loss and filler, foaming and adsorbent, chitosan has potential application in the medical fields. Chitosan, only in its purest form, has potential as an internal hemostatic dressing, as a drug delivery agent, as tissue scaffolding and numerous other health related products. However, as a natural product derived from the shells and components of organisms found in natural environments, chitosan carries a variety of contaminants, common to biologically derived materials, and thus must be carefully purified and its resultant stimulatory immune responses in humans and animals well understood before use in medically related applications. As shrimp is the most common source of chitosan, and shrimp live commensually with bacteria on their shells, bacterial endotoxin are one of its natural contaminants. See Nakagawa, et al., “Endotoxin Contamination in Wound Dressings Made of Natural Biomaterals” J. Biomed. Mater. Res. Part B: Appl. Biomatter, Vol. 66b, p. 347-355 (2003).

The ability to purify chitosan is further hampered by its ability to avidly bind endotoxins, including exogenous endotoxins from the environment. Because of the hydrophobic backbone, chitosan interacts with the lipid part of the endotoxin and its partially-positively charged amines interacts favorably with the negatively charged phospholipids on the endotoxin. Chitosan has been shown to reduce the apparent immunogenicity of endotoxin, but does not render it inactivate or destroy it. See Davidova, et al., “Determination of binding Constants of Lipopolysaccharides of Different Structure with Chitosan” Vol. 71, p. 32-339 (2006). Consequently, extended exposure to the chitosan-endotoxin complex can lead to long term inflammatory activity. The use of chitosan to reduce the activity of E. coli lipopolysaccharide, LPS, by binding the LPS was studied by looking at the cytokines produced. For chitosan and LPS-chitosan complexes, human mononuclear blood cells (wild type), human embryonal kidney cells (HEK293 transformed cells) and murine monocyte cell line RAW 264.7 were used to determine the production of IL-8 and TNF-α See Yermak et al., “Forming and immunological properties of some lipopolysaccharide-chitosan complexes,” Biochimie Vol. 88, p. 23-30 (2006).

As taught U.S. Pat. No. 6,699,386 to Todokoro et al, chitosan has been used as an endotoxin adsorbent and as a material for removing endotoxin from liquids and solutions as taught in U.S. Pat. No. 5,169,535 to Adachi et al., and U.S. Pat. No. 4,885,168 to Hashimoto et al. Hypotheses suggest that the hydrophobic lipid of the endotoxin associates noncovalently with the chitosan backbone. See Davydova, et al., “Interaction of Bacterial Endotoxins with Chitosan. Effect of Endotoxin Structure, Chitosan Molecular Mass, and Ionic Strength of the Solution on the Formation of the Complex,” Biochem (Moscow); Vol. 65, No. 9, p. 1278-1287 (2000).

Furthermore, under non-endotoxin-free conditions, chitosan continually absorbs endotoxin from the environment. U.S. Pat. No. 7,125,967 to Hung et al. teach the removal of endotoxin from chitosan to form a water-soluble chitosan by reacetylating soluble chitosan in a slightly basic solution that contains a phase transfer agent including quaternary ammonium salts, crown ethers and/or pyridinium salts and transferred in a variety of organic solvents. Molecular weight (MW) is degraded in the process. However, this method is dependant on organic solvent extraction which is cost prohibitive in large scale production of biomaterials. The utilization of solvents are prone to organic residues which are highly toxic for food or biomedical applications. Hung et al also fails to teach how to prevent contamination of the clean chitosan product. Additionally, the process presupposes a soluble chitosan; thus a low-molecular weight solvent soluble chitosan is required.

Few sources report methods for purifying chitosan once it is prepared. U.S. Pat. No. 6,898,809 to Qian et al. utilize metal and protein complexing agents to remove unwanted metal and protein contaminants from insoluble chitosan. Qian et al. disclose removal of toxins through a variety of pH changes, but fail to disclose techniques for removing endotoxins. U.S. Pat. No. 6,989,440 to Sannan et al. disclose a method of purifying chitin utilizing organic and organic acid solvents to remove fatty acids and salts including calcium. Takanori et al fail to disclose the removal of exogenous or endogenous endotoxins.

Removal of proteins, calcium carbonate and other major shell component contaminants are disclosed in processes for producing a clean chitosan from shells. Additionally, methods involving quenching in liquid nitrogen have been disclosed to minimize chemicals, with the addition of a dialysis step to produce “biocompatible” chitosan, such as in U.S. Pat. No. 6,310,188 to Mukherjee. However, none of these processes teach the significance of endotoxin levels or the relevant role of exogenous endotoxin in the final processes of chitosan drying or preparation.

In addition to the lack of suitable methods of producing medical and food grade endotoxin-free chitosan, little exists by way of testing the purity of the chitosan. As discussed above, pyrogenicity is a measure of the toxicity, lethality or inflammatory response to a wide range of biological materials that contact with blood and ultimately produce fever. Lipopolysaccharides from Gram-negative bacteria having this known activity are described as endotoxins. Lipopolysaccharides that do not induce this type of response are not considered endotoxins. The primary tests for validating the efficiency of depyrogenation procedures for endotoxin in particular include the USP rabbit pyrogen test or the LAL (limulus ameobate lysate) assay derived from horseshoe crabs. The LAL assay, both colormetric end point and kinetic or gel forming, has variable activity depending on the source and type of endotoxin as well as the source of crab.

The standard and FDA-approved assay for the presence of endotoxin is the Limulus amoebate lysate assay. This lysate produces a measurable response in the presence of available endotoxin. Unfortunately, this enzyme system is sensitive to polycationic materials, and thus can be invalid in the presence of chitosan. Consequently, LAL assays for testing medical grade chitosans use large dilutions and combinations of acidic buffers in an attempt to keep the chitosan solubilized and maintain enzyme integrity. For high enough concentrations of endotoxin, such as in food grade or commodity grade, this method of dilution works well—the chitosan is often diluted to the point where it does not interfere with the enzyme, and the original concentration of endotoxin per mass of chitosan is easily calculated by taking into account the dilution factors.

However, for the very low endotoxin chitosans (ultra-pure), this method no longer provides sufficient sensitivity. Furthermore, LAL is only a measure of the endotoxins present, and not a representative measure of the pyrogenicity, or the ability of the material, its other contaminants and innate ability, to produce an immunogenic response.

Further complications in the LAL assay can be noted. It is currently not known if the chitosan-bound endotoxins are active in the LAL assay. It is well known, in vivo, that circulating endotoxins are immediately bound by LPS-binding protein to create an bio-active form, but endotoxins may not be active in the chitosan-bound complex. Although variability is known in this assay, the FDA has approved an acceptable range of −50% to 200% variation in a known sample of control standard endotoxin (CSE) and allows for that range of variability in approving a material.

Simplifications to this assay have been made that take a single component of the LAL system known to bind to endotoxins and generating antibodies against them. U.S. Pat. No. 6,696,261 to Patel et al., disclose an in vitro pyrogen test where a pyrogen-free antibody to the cytokine is applied to a surface in a pyrogen-free assay system. This assay is more sensitive than the “two-plate” assay and detects endotoxins as low as 0.01 EU/ml. Most commonly, a peptide or peptide fragment from the LAL cascade is used. U.S. Pat. No. 6,645,724 to Ding et al. discloses the production of a recombinant Factor C protein from horseshoe crabs. U.S. Pat. No. 6,849,426 to Chen et al. discloses utilizing the protein to optimize a fluorescent surfactant that indicates the presence of endotoxin. U.S. Pat. No. 5,316,911 to Baek et al. discloses utilizing single components of horseshoe crab lysates to which an antibody is bound, avoids multiple steps. However, all of these LAL-based assays and the occasional rabbit based pyrogen tests are unpredictable in consistently ascertaining pyrogenicity of endotoxins in humans. The phylogenic distance between the horseshoe crab and higher vertebrates fails to accurately predict actual potency in mammals. See Nakagawa, et al., “Endotoxin Contamination in Wound Dressings Made of Natural Biomaterals,” J. Biomed. Mater. Res. Part B: Appl. Biomatter, Vol. 66b, p. 347-355 (2003).

The use of dendritic cells to study the expression of cellular response to pathogens is well known. Human monocytoid cell lines have been used to examine the pyrogen response of supernatants from bacterial samples. See Peterbauer et al., “Interferon-γ-primed monocytoid cell lines: optimizing their use for in vitro detection of bacterial pyrogens,” Journal of Immunological Methods, Vol. 233 p. 67-76 (2000); and Eperon et al., “Human monocytoid cell lines as indicators of endotoxin: comparison with rabbit pyrogen and Limulus amoeocyte lysate assay” al J of Immunological Methods, Vol. 207 p. 135-145 (1997). The use of human monocytes to validate or compare these cellular responses to endotoxin to the LAL, human whole blood culture or rabbit pyrogen test is also well known. See Hoffmann et al., “International validation of novel pyrogen tests based on human monocytoid cells,” J of Immunological Methods, Vol. 298, p. 161-173 (2005) and Nakagawa, et al., “Evaluation of the In Vitro Pyrogen Test System Based On Proinflammatory Cytokine Release From Human Monocytes: Comparison with a Human Whole Blood Culture Test System and with the Rabbit Pyrogen Test” Clinical and diagnostic Laboratory Immunology, p. 588-597 (May 2002). No standard assay currently exists for determining the cellular response of bound, loosely bound and slowly diffusing endotoxin from chitosan.

The present invention overcomes the drawbacks of the prior art and provides a method of making a low-endotoxin (ultra-pure) chitosan and an assay for testing in vitro the pyrogenicity of these chitosan based ultra-pure materials.

### SUMMARY OF THE INVENTION

It is, therefore, an objective of the present invention to provide a method for processing commodity or food grade chitosan to form an ultra-pure, low endotoxin, chitosan product having medical and biological applications.

It is also an objective of the present invention to provide a method for processing chitosan, extracted from shells to form an ultra-pure, low endotoxin chitosan product having medical and biological applications.

It is also an objective of the present invention to provide an assay method for determining pyrogenicity in chitosans.

These and other objectives are described hereinbelow.

### DESCRIPTION OF THE DRAWINGS

FIG. 1 shows a sketch of endotoxin with specific saccharide residues from E. Coli.

FIG. 2 shows the molecular structure of an LPS from E coli.

FIGS. 3(a) and 3(b) show endotoxin unit (EU) levels measured by Limulus amoebate lysate (LAL) assay. FIG. 3(a) shows the measured EU/mL for the 4 positions of material above the filter for samples of processed material. FIG. 3(b) shows the determined EU/g of chitosan for the 4 positions of material above the filter of processed material at two different dilutions as well as the food grade starting material, acid and water control.

FIG. 4 shows endotoxin levels measured by Limulus amoebate lysate (LAL) assay after each processing steps from three samples of shrimp shells.

FIG. 5 shows the TNF-α response in pg/mL of primary human macrophages after exposure to food grade chitosan.

FIG. 6 shows the TNF-α response of various subsets of human monocytes (THP-1) in pg/mL after exposure to food grade chitosan.

FIG. 7 shows the TNF-α response of various subsets of human monocytes (THP-1).

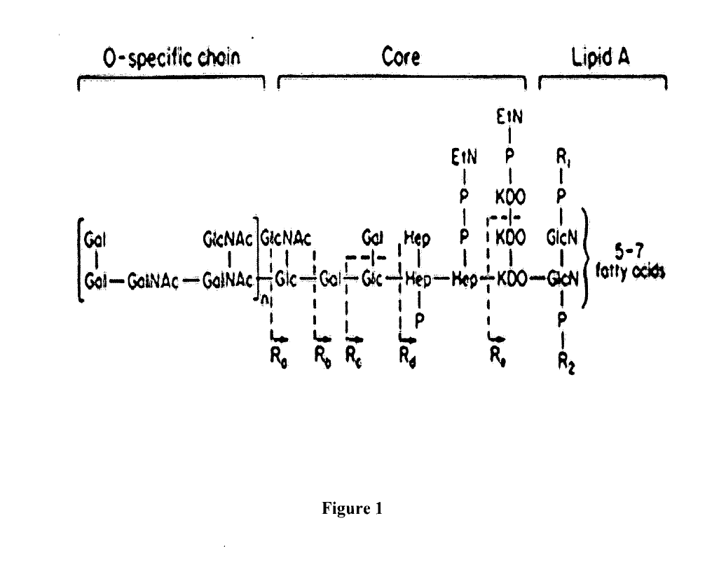

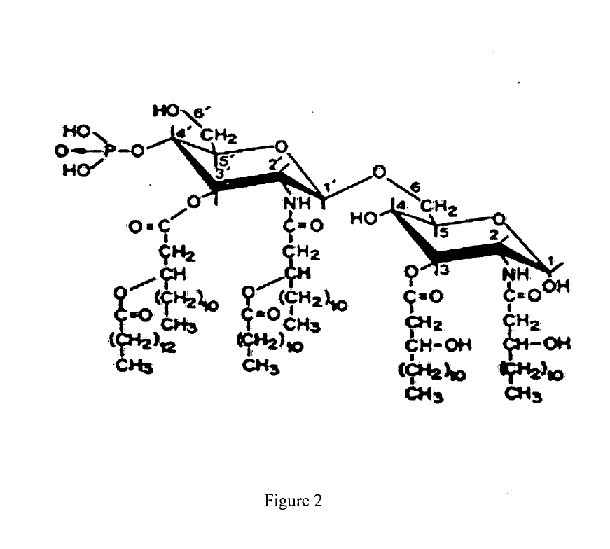

### DETAILED DESCRIPTION OF THE EMBODIMENTS

Given its natural predilection towards minimal toxicity and biological inertness, chitosan can be a very important component of medical and biological technologies. Although commodity grade chitosan is inexpensive and abundant, it has remained unsuitable for non-food applications in humans and animals due to residual natural contaminants, primarily protein and endotoxins. In food applications, these residual natural contaminants are easily passed through the digestive tract without harboring ill effects.

In medical and biological applications involving open wounds, blood and organs, the available endotoxins in non-medical grade chitosan initiate innate and adaptive immune responses that render chitosan unsuitable for medical use. In recognition, the United States Food and Drug Administration has imposed strict guidelines on the allowable levels of endotoxin in pharmaceuticals, medical devices and products that contact human tissue, blood, bone or that can be absorbed by the body or implanted within the body.

Current methods of producing medical grade (ultra pure) chitosan are limited to low molecular weight chitosan and/or are cost prohibitive. Furthermore, these known methods fail to provide means for retaining purity throughout processing and upon storage. Purity retention and molecular weight retention, particularly for high molecular weight chitosan, is a challenge, because of chitosan's ability to absorb and bind exogenous chitosan from the environment, both during processing and storage. There is a clear need for inexpensive methods to produce and maintain high molecular weight, ultra-pure chitosan. There is also a clear need for cell-based assays to rapidly and consistently determine the suitability of the ultra-pure chitosan for its respective uses.

It is important to note that the preservation and control of MW is critical to the present invention. Tissue scaffolding and cell support require chitosan of a substantial MW, greater than 30 kDa, as do wound dressings and drug delivery systems. While other processes do not require chitosan of specific MWs or require low MW chitosan for solubility during the cleaning and processing stage, the present invention maintains the molecular weight of the chitosan within 75% to 90% of the original value.

It is also important to note that the present invention is not directed to the actual production of chitosan. Rather, this application is directed to creating, optimizing and maintaining chitosan purity. The description of the methods in accordance with the present invention follow.

Medical Grade Chitosan from Commodity or Food Grade Chitosan

The present invention is directed to the production of high quality, medical grade chitosan having ultra-low endotoxin levels (hereinafter referred to as LoTox chitosan) and the LoTox chitosan produced therefrom. It is important to note that the endotoxin levels in the LoTox chitosan are in the range of 100 EU/g to 20 EU/g. The lower limit cannot be quantified by methods at present. Given the challenges in current LAL technologies, the limit of detection is approximately 20 EU/g of chitosan. Lower values are attainable in accordance with the present invention. However these lower values cannot be detected. In a preferred embodiment, the LoTox chitosan is produced from commodity or food grade chitosan, while retaining molecular weight integrity without the use of organic solvents, phase transfer catalysts or chemical modification to the chitosan. Because organic solvents can have toxic reactions in the body, their absence is very important.

In accordance with the present invention, commodity or food-grade chitosan is contacted with a strong base at elevated temperatures. This step destroys the significant fraction of the endotoxin present in the material, regardless of the source, which can then be rinsed away. In a preferred embodiment, dry or pre-swelled chitosan is contacted with ultrapure, pyrogen-free water for up to 24 hours or pre-swelled in up to 10% methanol in ultrapure water. This formulation is then treated in 0.1 to 1.0 M NaOH at 80-90° C. for 90 min. Higher concentrations of base may be used, however the integrity of the molecular weight is compromised. The molecular weight of chitosan rapidly at high temperatures for [OH—]>0.1M.

In accordance with the present invention, it is crucial to control several variables integral to the present process of endotoxin removal from commodity or food grade chitosan. The base breaks the phosphodiester bond between the lipid and polysaccharide part of the LPS. However its action must be controlled to prevent the systematic break down of the glycosidic link between the glucosamine rings in the chitosan backbone. Controlling reaction temperature is critical in optimizing the purity of the LoTox chitosan. Controlling concentrations of the starting material is also of importance, but can be overcome by utilizing significantly longer reaction times and higher temperatures, provided that the concentration of NaOH is above 0.01M. The correlation of temperature (exponentially dependant) and concentration (linearly dependant) to the optimal purity is well understood to one of ordinary skill. Stirring is also required to keep the chitosan particles from settling and providing full surface area contact with the solution. Smaller particle sizes of chitosan are more optimally cleaned. Because chitosan is insoluble in base, it remains in a particulate form. Hence, the initial step of preswelling in water or methanol helps to solvate the chitosan and provide a larger surface area for exchange of solution and endotoxin.

In a preferred embodiment, the method utilized in producing LoTox chitosan from food grade chitosan is described below. Chitosan with MW greater than 30 kDa is insoluble at physiologic pH (in the range of 6.8 to 7.5). In accordance with the present invention, the MW of LoTox is maintained above 30 kDa, and within 10% to 25% of the MW of the starting material. It is important to note that chitosan decomposes in base (e.g. NaOH) at high temperatures. Typically, decomposition begins at approximately 4 hrs at 90° C. with 1.0M NaOH. Thus, very large decreases in MW are not accessible with this technique.

In accordance with the present invention, all of the steps described below utilize pyrogen-free labware, reagents and materials and are conducted in a strict endotoxin-free environment:
1) Food grade chitosan is swelled up to 24 hours in pyrogen-free water or a methanol ultrapure water solution, alternatively, dry food grade powder is prepared;
2) The food grade chitosan of step (1) is then dissolved in a hydroxide base and continuously stirred. In a preferred embodiment, 1 kg/25 L to 1.5 kg/25 L (requires a large processing vessels for optimal results) ratio of the chitosan is dissolved in 0.01 M to 4.0 M NaOH and continuously stirred, where the preferred molar concentration is 1.0 M of NaOH;
3) The chitosan base solution of step (2) is stirred and heated between 60° C. and 100° C. for 45 minutes to 4 hours (longer processing times produce lower molecular weight end product), where optimal results are obtained at 90° C., temperatures below 60° C. are not effective, where a 1 hour processing time results in a <15% reduction in MW at 1.0 M NaOH, and where the resultant chitosan at 1.0M and 90° C. has <20 EU/g;
4) The processed chitosan solution is rinsed with up to 10× volume of ultra-pure pyrogen-free water to remove endotoxin fragments and residual endotoxin, where rinsing to neutrality (pH between 6.8 to 7.5) is sufficient;
5) The resultant slurried chitosan is transferred in a closed system (slurried LoTox chitosan can be easily pumped when it is 25% weight in water) without exposure to endotoxin;
6) The water may be removed by filtration, aspiration or decanting; and
7) The resultant LoTox chitosan is stored in storage containers in an endotoxin-free environment with sterile, endotoxin-free connects.
   The LoTox (ultra-pure) chitosan may be shipped as a paste (˜11% by weight) or in water at any chitosan:water ratio in endotoxin-free containers.

Table II shows the effect of processing time and temperature for a 1.0M NaOH concentration on the resultant MW of LoTox (ultra-pure) chitosan after processing food grade chitosan. As noted above, longer processing times at 1.0M base (NaOH) concentrations decreases the molecular weight more for longer periods of time at this high NaOH concentration (1.0 M). It is however, important to note that temperature variations, provide a more dramatic effect. Thus, as noted above, temperature is the more important variable for preserving molecular weight. It is also critical that all processing must occur in pyrogen-free labware under strict endotoxin-free environment. Endotoxin levels were similar within the variability of the limulus amebocyte lysate assay for endotoxin (LAL).

TABLE II
Properties of medical grade chitosan for
processing conditions at 1.0M NaOH

| Temperature ° C. | Time (min) | NaOH (M) | MW* | % change |
|---|---|---|---|---|
| Chitosan | | | 370500 | |
| 80 | 45 | 1.0 | 282800 | 22 |
| 80 | 60 | 1.0 | 274700 | 24 |
| 60 | 45 | 1.0 | 310600 | 14 |
| 60 | 60 | 1.0 | 302900 | 16 |

*Note that dn/dc values used were not measured for each sample but were taken from the literature for pure chitosan at 85% deacetylation thus the MW vales in Tables II through IV are comparative.

Table III shows the effect of NaOH concentration at 90° C. on the resultant MW of medical grade chitosan after processing food grade chitosan for 120 minutes. A molecular weight change of 18% is observed with the 0.01M NaOH, while at the higher concentration, a 43% decrease in molecular weight is observed. Depending on the desired final molecular weight, the NaOH concentration can be varied with very little effect on measurable endotoxin levels for this time and temperature of processing.

TABLE III
Properties of medical grade chitosan after various
processing conditions at 90° C.

| Temperature | Time | | | | |
|---|---|---|---|---|---|
| ° C. | (min) | NaOH (M) | MW* | Average | % change |
| Chitosan | | | 370500 | | |
| 90 | 120 | 0.01 | 321400 | 305650 | 18 |
| 90 | 120 | 0.01 | 289900 | | |
| 90 | 120 | 0.1 | 279400 | 273050 | 26 |
| 90 | 120 | 0.1 | 266700 | | |
| 90 | 120 | 1.0 | 221000 | 211700 | 43 |
| 90 | 120 | 1.0 | 202400 | | |

*Note that the MW is based on dn/dc values determined from chitosan alone

### Example 1

### Molecular Weight Dependence on Processing Variables

Depyrogenated labware (all glass or stainless steel, baked at 250 C for 1 hrs (to <<0.1 EU/g of material or rinsed water), depyrogenated water (<0.005 EU/mL) and sterile procedures are utilized for all processing steps. All processing is done in a class 1000/10000 clean room using sterile procedure. Food grade chitosan, approximately 100 μm in diameter and having a MW of 326900 Da, was treated by stirring in 1.0M NaOH at 90° C. for 60 minutes at a ratio of 0.2 g/20 mL and rinsed with 500 mL of endotoxin-free water (<0.005 EU/mL). The resultant LoTox chitosan had endotoxin levels measured by LAL to be <20 EU/g and a MW reduction shown in Table IV. This table shows the variability in MW for different treatments and volumes and is within the reproducibility of the MW measurement for broad MW peaks of 10%.

TABLE IV
Properties of medical grade chitosan Lotox product

| Temperature ° C. | Time (min) | NaOH (M) | Mw | % change |
|---|---|---|---|---|
| Chitosan | | | 326900 | |
| 90 | 60 | 1.0 | 299700 | 8 |
| 90 | 60 | 1.0 | 277100 | 15 |
| 90 | 60 | 1.0 | 279000 | 15 |

### Example 2

### Ultra-Pure Chitosan Reaches the Limits of Detection of Standard LAL Assay

LoTox chitosan (LoTox) was prepared by dissolving 75 g of food grade chitosan in 940 mL of 1M NaOH at 90 C for 1 hour. Two 75 g batches were synthesized. The material was transferred in a class 10000 cleanroom under sterile conditions to pyrogen-free filters and rinsed with approximately 4 L of endotoxin-free water until the pH was approximately 7.5. The chitosan paste was measured to be 11% chitosan by weight. This material was tested for endotoxin levels by LAL assay.

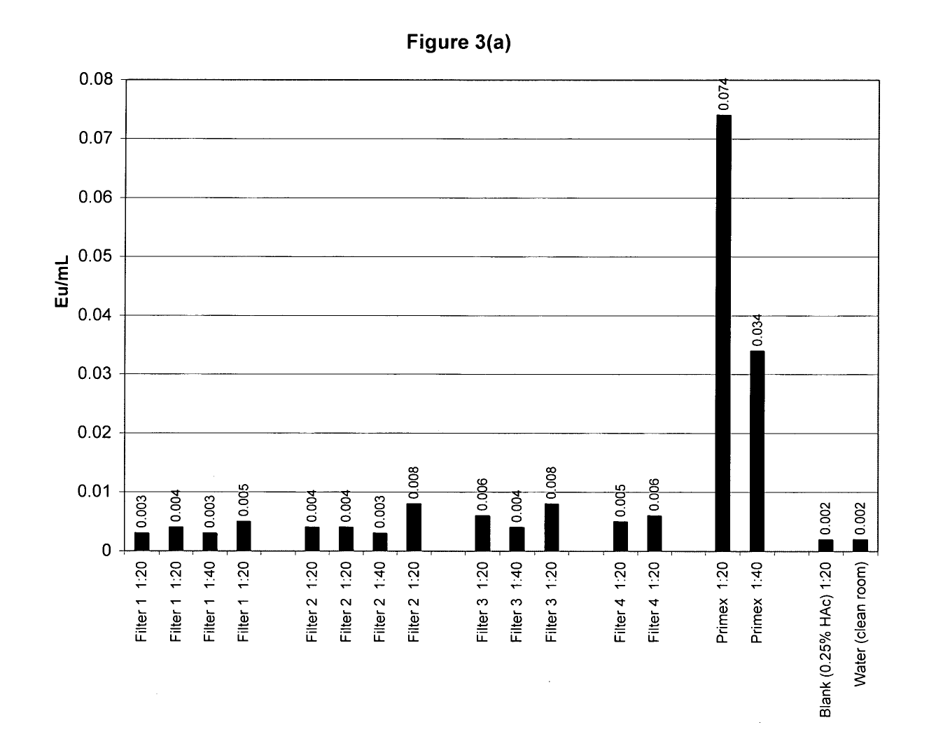

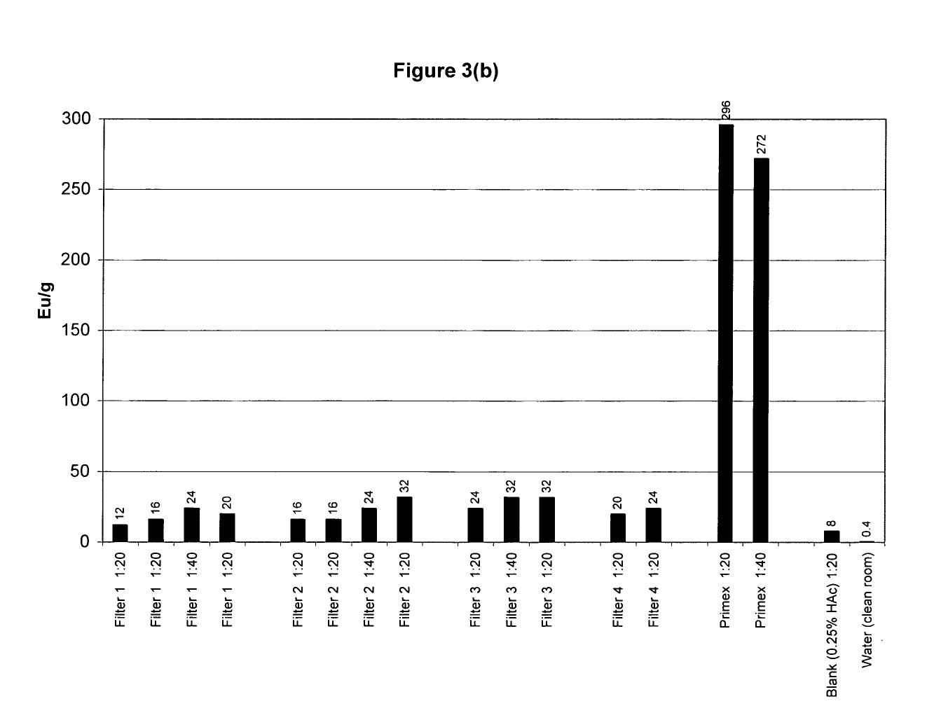

Food grade chitosan was processed by treatment in 1M NaOH at 90° C. under the conditions described above preserving both a sterile and pyrogen-free environment. The material was transferred to a sterile, pyrogen filter and measurements of endotoxin were made by the method of dilution in weak acid in a standard LAL kit. FIGS. 3(a) and 3(b) shows measured endotoxin levels in EU/mL as measured and in EU/g as calculated from the mass of chitosan in the solution. As shown in FIG. 3(a), dilutions of 1:20 and 1:40 of the original 0.25% acetic acid solution with 0.5% by weight chitosan are shown for chitosan samples taken from the top, 2^{nd} layer, 3^{rd} layer and bottom (surface of the filter). First, the controls of food grade chitosan (Primex) show the expected ˜2-fold decrease in EU/mL for a 2-fold dilution (0.074 to 0.034 EU/mL). However, the dilutions do not show the expected two-fold decrease in EU/mL for the ultra-pure material. For example, a 1:20 dilution measuring 0.003 EU/mL should measure 0.0015 EU/mL when diluted to 1:40. However, the values are unchanged upon dilution. This result shows that the limits of detection of the assay have been reached. Second, all of the layers show that the position of the material in the filtration process does not affect the final endotoxin value (as it can be determined below the limits of detection) as evidenced by the same EU/mL measurements for all of the layers. FIG. 3(b) shows the calculated EU per gram of LoTox chitosan. Note that for the control with food grade material, the final values are close (296 and 272 EU/g) as expected. However, the calculated EU/g increases for the 1:40 dilution for the same sample, again showing not an increase in endotoxin, but the limits in using this technique to measure reliable EU/g for samples less than about 50 EU/g.

Medical Grade Chitosan Production from Shells

There are several established protocols for chitosan production, and each of those produces variable grades of endotoxin-laden material. As is understood by one of ordinary skill, the methodology of the present invention is directed to specific, novel and unobvious steps that are to be utilized in the chitosan production process to produce a ultra-low endotoxin chitosan.

A preferred embodiment of the present invention incorporates controls in the step of processing the chitin with a strong base. This step destroys the available endotoxin present in the material, regardless of the source, and is rinsed away. In a preferred embodiment, the present invention identifies that the critical point for producing an ultra-low endotoxin chitosan is just prior to deproteination/deacetylation (and any step that involves very strong base pH>10). In another preferred embodiment depyrogenated labware (all glass or stainless steel, baked at 250 C for 3 hrs (to <<0.1 EU/g of material or rinsed water) were utilized, depyrogenated water (<0.005 EU/mL) was used and endotoxin-free procedures for all processing was utilized. All processing was done in a class 1000/10000 clean room, as discussed below.

In accordance with the present invention, ultra-low endotoxin chitosan is produced utilizing the following methodology. As above, sterile, pyrogen-free labware, reagents and materials were utilized:
1) Endotoxin levels, whether endogenous or exogenous, are not of primary importance prior to the deproteination step (if that step is subsequently followed only by the deacetylation step);
2) The deproteination and deacetylation steps can be combined, and previous steps are not important in assuring endotoxin levels in chitosan
3) At the point of deproteination (and before deacetylation) the whole process including milling or grinding is performed in an endotoxin-free environment; In another preferred embodiment, the endotoxin-free environment is a class 100 cleanroom (as is understood by one of ordinary skill in the art). For lesser quality chitosan products, a class 1000 to 10000 cleanroom is sufficient. Alternatively, small test batches may be handled in an endotoxin-free laminar flow hood under strict endotoxin-free conditions;
4) All materials and chemicals are depyrogenated or purchased pyrogen-free. This includes but is not limited to: water, NaOH (or other strong base including but not limited to KOH) beakers, flasks, measuring devices, stirring devices, filter paper, filter cloth, stainless steel production vessels, glass vessels, transfer vessels, storage vessels, air and vacuum canisters (and any other device, container or vessel with which the product will come into contact); Note that sterility does not equal or imply pyrogen-free as noted above.
5) All processing is done either remotely or automatically or by personnel trained in ultra endotoxin-free handling processes;
6) Deproteination and deacetylation are carried out under the strictest of endotoxin-free and sterile conditions, particularly in the handling and rinsing of the final product(s) using a strong hydroxide base including, but not limited to, NaOH or KOH. Endotoxin within the mixture is destroyed in the processing. The final processing step (the deacetylation) removes most of the remaining and minimal endotoxin from the previous deproteination step;
7) If any endotoxin is present or introduced during the rinsing of the product after the deacetylation step, it remains in the chitosan. Thus, all residual endotoxin must be removed before (during deproteination) or during the deacetylation steps; and
8) Packaging, handling and storage of the product is performed in the same manner of an ultra endotoxin-free, sterile environment. All bags, vials, containers, boxes, air handling, sealing and storage materials are pyrogen-free. Product is stored in air-tight, impermeable, endotoxin resistant and pyrogen-free containers.

The steps of the present invention provide a novel and unobvious LoTox chitosan product (after deacetylation) having an endotoxin concentration that is routinely less than 100 EU/g for ground chitosan. The method of the present invention can be utilized to provide ultra-low endotoxin chitosan having endotoxin concentrations <20 EU/g routinely for particles averaging 100 μm in diameter or less.

In a preferred embodiment, the final step of deacetylation (high concentration of base, elevated temperature) is the final step and the point at which endotoxin-free procedures and containers are required.

The method of the present invention also provides a LoTox chitosan that is free of any residual exogenous chemicals and does not require the use of detergents (e.g. Triton X100 or Triton X114), amphiphilic solvents or phase-transfer solvents to remove endotoxins. In a preferred embodiment, the method of the present invention does not alter the molecular weight of the material produced nor any other physical characteristic other than the unwanted introduction of exogenous endotoxin.

In accordance with the present invention the LoTox chitosan includes hydroxide, chloride and sodium, standard processing components and the minimal endogenous materials. As is understood by one of ordinary skill, other salts may be present if other acids or bases are used. However, no organic solvents are present because each of the steps discussed above are conducted in aqueous solution. Because organic solvents can have toxic reactions in the body, their absence is very important. In this manner, the ultra-low endotoxin chitosan product of the present invention maintains the integrity of its molecular weight, as the chitosan as prepared is ultra-pure. No post processing in required.

The product formed is rinsed copiously with ultra-pure water and is stored as a damp final product. Unless dried in sterile, endotoxin-free processing, such as pharmaceutical grade lyophilization or pharmaceutical quality vacuum drying, the chitosan will pick up exogenous endotoxin. Thus, for maintenance of the ultra-pure levels of endotoxin in chitosan, minimal and only highly controlled post processing can be done.

It is also important to note that the method of the present invention provides ultra-low endotoxin chitosan products having endotoxin levels below 100 EU/g and levels routinely below 20 EU/g, depending on the length and temperature of the deacetylation step. In another preferred embodiment, the methodology according to the present invention can be used with any processing technique that uses any hydroxide base process as its last steps (KOH, NaOH, etc) and indicates the critical point in a processing streamline after which endotoxin contamination must be eliminated, ultra-clean methodology must be followed and strict environmental controls must be maintained. If grinding is to occur, it must be before the processing unless done in pyrogen-free, environment in the absence of ambient air that contains endotoxin.

### Example 3

### Dirty and Clean Shells have Similar Endotoxin Levels after Deproteination

Three shell preparations from the same source were tested using standard literature processing conditions 1) dirty shells cut into large chunks; 2) dirty shells ground into a powder with maximum dimension ˜1-2 mm; and 3) a standard clean shell ground to 0.5 mm max dimension. Dried shells were tested by overnight shaking in depyrogenated water at room temperature followed by the kinetic limulus amebocyte lysate assay for endotoxin (LAL). Demineralization proceeded through the use of a 1M solution of HCl, with a 1:10 (w/v) ratio of dried shrimp shell to aqueous acid. This solution provides for a molar excess of greater than 2.5 equivalents. The vacuum dried shells (37° C. for 15 hours), ground or chopped, are massed (2 g) and reacted in a suitable pyrogen-free Erlenmeyer flask equipped with a stirbar. The heterogeneous solution is stirred for 30 minutes and the liberation of gas can be observed. After this time, the solution is filtered and washed to neutrality with pyrogen-free water. An aliquot of shells is removed for endotoxin testing. The demineralized shell is placed in a depyrogenated round bottom flask equipped with a stir bar and 20 mL of 1 M NaOH is added. This volume provides an approximate 1:10 w/v ratio. This heterogeneous solution is placed in a constant temperature oil bath (90° C.) and stirred for 2 hours. The solution is then filtered and rinsed to neutrality with pyrogen-free water. An aliquot of the filtered shells is removed for endotoxin testing. For one subset of standard clean shells, 2500 EU (endotoxin units) of exogenous endotoxin were added before the demineralization step (BM) and the processing as described above followed. For another set of standard clean shells 2500 EU exogenous endotoxin were added before the deproteination step (BP) and the processing as described above followed.

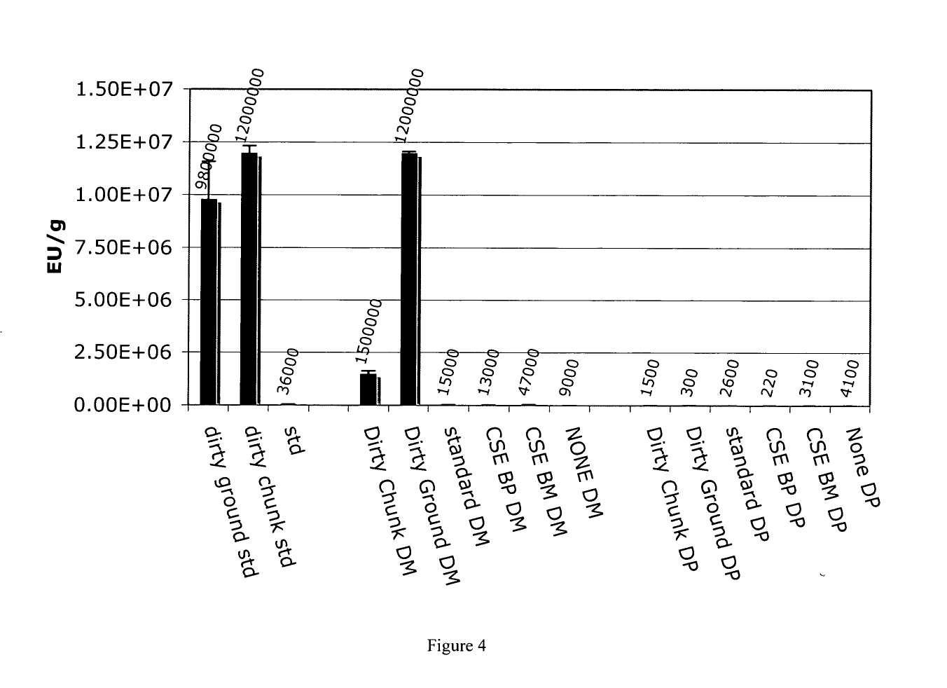

FIG. 4 shows the endotoxin levels measured by LAL assay (EU/g) after each of the processing steps from the three different samples of shrimp shells. Two standard curves of endotoxin with two separate batches of endotoxin confirm the validity of the numbers measured to +/−<10%. FIG. 4 also shows that unprocessed dirty shells have very high residual levels of endotoxin (˜10⁷ EU/g) whereas the unprocessed “clean” shells have ˜10⁴ EU/g. Additionally, FIG. 4 highlights the relative residual endotoxin levels after demineralization (DM) and after deproteination (DP). Note that in the “NONE” sample, the standard clean shells were subjected to the same processing times and temperatures, but with only water and no chemicals. Endotoxin levels decrease slightly or increase after demineralization, an effect that is considered to be a result of the exposure of internal endotoxin by the demineralization process. After the deproteination step, endotoxin levels decrease dramatically, up to 5 orders of magnitude in some cases. In fact, even for the largest contamination on shells, the resultant product after deproteination is similarly clean, within a factor of 10. More importantly, the processed very dirty shells were as clean, or cleaner, even though they contained endotoxin levels many orders of magnitude greater than other shells even after the demineralization step. This result shows that the quality of the shells up to the point between demineralization and deproteination has no effect on the final quality in terms of endotoxin on the final product. Note that the residual levels of EU may also be enhanced due to contamination post processing when the materials were removed from the cleanroom and shaken in an incubator overnight. Furthermore, the HCl and NaOH were not pyrogen-free nor were the containers in which they were prepared or stored

### Cell-Based Assay for Determination of Pyrogenicity of Chitosan

Rapid and in vitro determination of the pyrogenicity of chitosan in humans and mammals is difficult because (a) chitosan is a polycation and interferes with the accepted LAL assay; (b) chitosan avidly binds endotoxin, establishing a bound-unbound equilibrium in solution; (c) accepted LAL assays do not necessarily reflect the pyrogenicity of endotoxins found in chitosan sources such as shrimp and (d) accepted LAL assays do not measure other pyrogenic biomaterials, such as Gram-positive bacteria. For example LAL assays cannot detect Staphylococcus aureus which is pyrogenic (as detectable through TLR2). These factors make the quantification of endotoxin in chitosan difficult. In addition, chitosan may have other immuno-stimulatory contaminants such as proteins, other bacteria, viruses and fungi. The effect of endotoxins as well as the cumulative effect of all pyrogens need to be determined for a medical grade material.

The pyrogenicity of soluble polycations, such as those disclosed in the inventors' application Ser. No. 11/657,382, filed Jan. 24, 2007 and incorporated herein by reference, are difficult to measure utilizing the LAL assay. As these polycations are synthesized under strict pharmaceutical standards, they inherently have low endotoxin levels (below measurable amounts). These polycations are soluble at and above physiologic pH (approximately 7) and highly positively charged. Due to their low endotoxin levels, they dramatically interfere with LAL enzymes and cannot be diluted. Finally, these molecules present endotoxin to LAL differently as they will tightly bind residual endotoxins. Thus, a cell-based response to these compounds/molecules are essential for understanding their potential pyrogenicity in mammalian and human systems.

Any source of dendritic cells that respond to pyrogens through the innate immune receptors can be used in the assay, although human cells are preferred. Cell lines of monocytes (such as THP-1 or Mono-Mac-6) can be used if primed by standard techniques and will have less variability than primary cell lines. However, primary cells tend to give a more representative distribution of the variability in human responses.

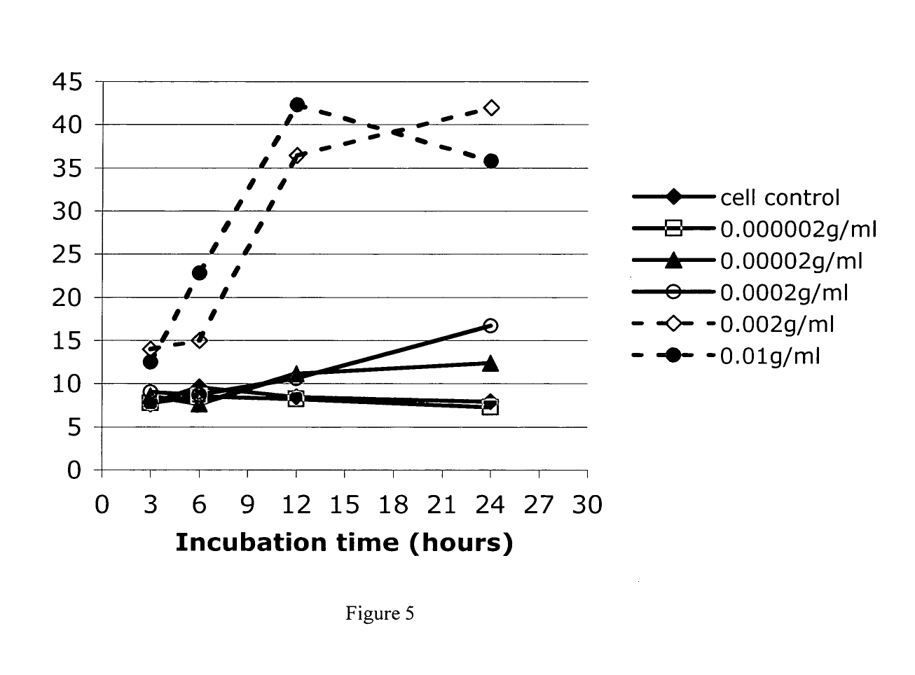

The assay is performed in the following manner. All materials, reagents and labware utilized are sterile and pyrogen-free. Human dendritic cells are grown in standard cell culture media with pyrogen-free serum and primed, as described for a chosen cell line activation, following standard biological procedures, THP-1 monocyte cell line or primary monocytes are preferred rather than macrophages that take up to 7 days to become confluent. In a preferred embodiment the cells are primed with calcitriol or interferon-γ and cell lines are used for two to three months and kept for no more than four months before a new culture is used. Primary cell lines are used immediately. Chitosan or other biologically derived material is delivered to equivalent wells of dendritic cells in replicates sufficient for statistical analysis. Doses of chitosan are <2 μg to 20 mg/mL where viability is decreased due to blanketing the cells with chitosan. A <2 mg/mL dose of chitosan is preferred. A control of food grade chitosan is used to validate the cells positive response to chitosan-presented endotoxins as well as media controls and cell negative controls. Bracketing the predicted response with known pyrogens is used to standardize the assay based on desired endotoxin values or other pyrogen values. For example, control standard endotoxin is administered at <0.01 EU/mL and at 10 EU/mL to bracket the variable cellular response. For a direct comparison with a particular cut-off value for acceptable endotoxin or pyrogen levels, that control should also be used. Note that the cellular response to endotoxin is not linear for macrophages and that once a threshold concentration is exceeded, the response becomes great as shown in FIG. 5. Media is taken from the wells at time points desired for an indicated response. For IL-6, IL-1β and TNF-α, the preferred time is 1 hour for monocytes although 3 and 6 hours is illustrative. Anti-inflammatory cytokines are typically a secondary response post inflammation due to pyrogens and will arise in 12-24 hours. However, rapid anti-inflammatory responses such as IL-10 and IL-4 can be induced by certain regulatory materials in the same time frame as the inflammatory responses. Media can be immediately frozen for later testing or in a preferred embodiment, tested immediately on ELISA plates of cytokine antibodies to quantify the mass of cytokine per mL of solution. Note that cytokine expression is transient and that the presence of cytokines is not cumulative as proteases and cellular binding and absorption mitigates the lifetime of proteins in solution. The pyrogen response of longer term exposure to chitosan and other materials can be determined in the same fashion but by continuing to culture the cells in the appropriate environment for the duration of the time course. In a preferred embodiment 12-18 hours is tested. Macrophages require a longer response than do monocytes and are measured at 12-24 hours.

### Example 4

### Response of Primary Human Macrophages to Different Doses of Food Grade Chitosan

The response of a primary cell line of human macrophages to food grade chitosan was measured as a function of time over a period of 24 hours. Macrophages were grown to confluency with macrophage stimulating factor and cultured in RPMI 1640 w/L-glutamine and sodium bicarbonate, 5% FBS heat inactivated low pyrogen, and 1% antibiotic/antimycotic. Dry powdered food-grade chitosan (Primex: 10.0, 2.0, 0.2, 0.002 and 0.002 mg/mL) was added to the media and samples were taken at 3, 6, 12 and 24 hours and tested in a standard TNF-α ELISA immunoassay 96 well plate. The time course is shown in FIG. 5. Note that the response of the cells is minimal up to 0.0002 g/mL (0.2 mg/mL) but respond dramatically and with little difference above 0.002 g/mL (2 mg/mL). This data indicates that the response to endotoxin as presented by chitosan is nonlinear, and a particular threshold is required to stimulate a large response over this time period.

### Example 5

### Comparison Monocyte Response to Food Grade Chitosan, Different Control Standard Endotoxins and Ultra-Pure Chitosan

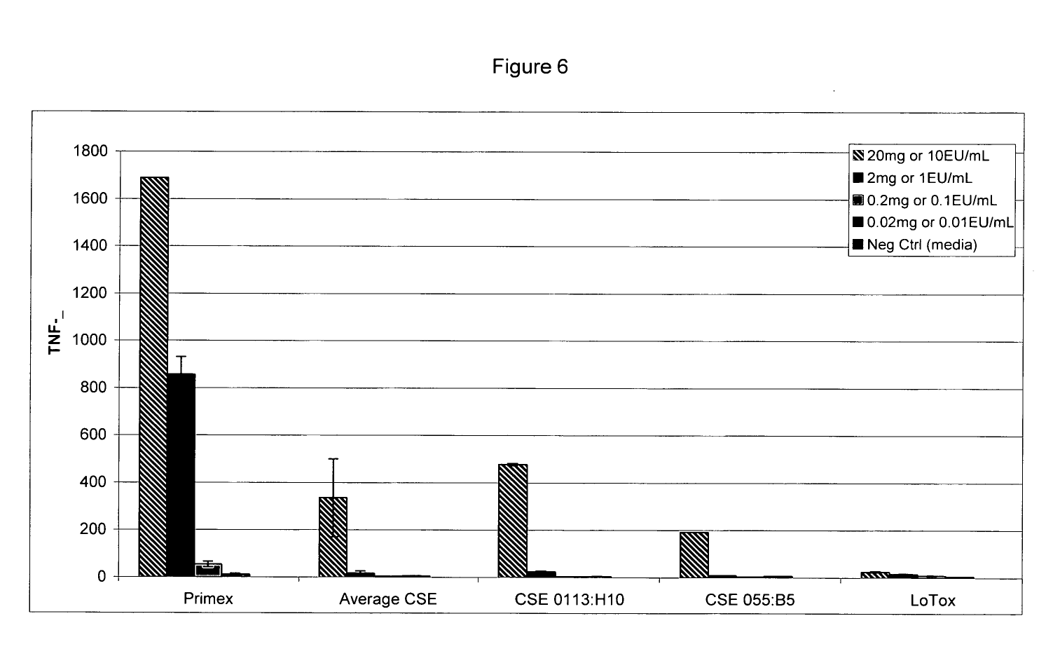

The LoTox chitosan was tested in a cell-based assay for its immune stimulating activity. The assay was performed to determine the TNF-α response of primed monocytes (THP-1 cell line, ATCC) to various concentrations of food grade chitosan, endotoxin from two different E. coli strains O113:H10 and O55:B5 alone and mixed equally and to ultra-pure chitosan. FIG. 6 shows the TNF-α response in pg/mL at pH 7 of 400,000 THP-1 cells are grown in RPMI-1640 based culture medium containing 10% serum, and primed for 44-48 hours before use with 10 ng/mL calcitriol. Media is tested after 1 hour exposure to the indicated stimulants. The food grade chitosan and ultra pure chitosan (Primex and LoTox) were presented to the cells in doses of 20, 2.0, 0.2 and 0.02 mg/mL while the endotoxins were presented in doses of 10, 1.0, 0.1 and 0.01 EU/mL. The control with media has a TNF-α response too low to be seen on this graph. As expected, a clear dose response is seen for all materials. Food grade chitosan having between 500-1000 EU/g of endotoxin stimulates significant TNF-α response equivalent to more than 10 EU/mL of control standard endotoxin. 10 EU/mL is also sufficient to induce cell death. Note that the cellular response from the ultra-pure chitosan (LoTox) at even the highest concentration is lower than the response of the endotoxin (CSE O113:H10) at 0.1 EU/mL.

### Example 6

### Panel of Cytokine Responses of Human Monocytes Exposed to Food Grade Chitosan, LoTox Chitosan, Endotoxin and Controls after 24 hours

Human monocytes were primed and grown as described in example 3, above. These cells were exposed to food grade chitosan, processed chitosans (Lots I and II) where the container was not closed and exogenous endotoxin could be absorbed. Endotoxin, cell only and media only controls at concentrations are shown in Table V. ELISA measurements were done for media based samples taken at 24 hours after exposure and shown in pg/mL of measured cytokine, also shown in Table V. The cytokines tested were pro-inflammatory (TNF-α, IL-1β, IL-6) and anti-inflammatory (IL-4 and IL-10). For the food grade chitosan the TNF-α and IL-1β well exceeded the assay capability and are most likely greater than the reported number. Likewise, many of the anti-inflammatory cytokines measured for all material (except food grade chitosan) were below the limit of detection of the assay. All numbers above or below the limits of detection are shown in italics. This data shows that clean chitosan only minimally stimulates pro-inflammatory cytokines and that a full-panel screen can help to understand the relative pyrogenicity of produced chitosan samples. It is important to note, however, that cytokines have limited lifetimes in solution and are degraded by proteases and taken up by receptors on cells. As such, these are instantaneous measurements of the cytokine response in solution at the time they were taken. It is also important to note that at 2 hours, anti-inflammatory cytokines begin to be stimulated which is part of the natural process of down-regulating an inflammatory response. This is seen here in the 24 hour data but not observed in 1 hour data where the anti-inflammatory response has not been instigated. It should also be noted that only the shaded cells in the table are statistically different from the controls.

TABLE V
Pro- and anti-inflammatory cytokines measured after 24 hour
exposure to tested chitosans, endotoxin and controls.

| | Pro-Inflammatory | | | Anti-inflammatory | |
|---|---|---|---|---|---|
| Material | Cytokines | | | Cytokines | |
| (amount/ml) | TNF-α | IL-1β | IL-6 | IL-4 | IL-10 |
| Food grade | 885.32 | 880.65 | 53.16 | 1.15 | 7.78 |
| (20 mg) | | | | | |
| Citosan Lot I (20 | 48.98 | 242.16 | 2.15 | 1.04 | 2.46 |
| mg) | | | | | |
| Chitosan Lot II | 26.29 | 149.17 | 2.14 | 1.04 | 1.63 |
| (20 mg) | | | | | |
| Endotoxin | 58.73 | 19.70 | 6.33 | 1.01 | 1.87 |
| (10 EU) | | | | | |
| Cells in Medium | 3.72 | 2.62 | 1.78 | 0.94 | 1.98 |
| Medium only | 0.03 | 0.02 | 0.02 | 0.01 | 0.02 |

### Example 7

### Comparative Measurement of TNF-α Response of Human Monocytes to Food Grade Chitosan, Endotoxin Controls and Ultra Pure Chitosan for Six Different Cell Lots

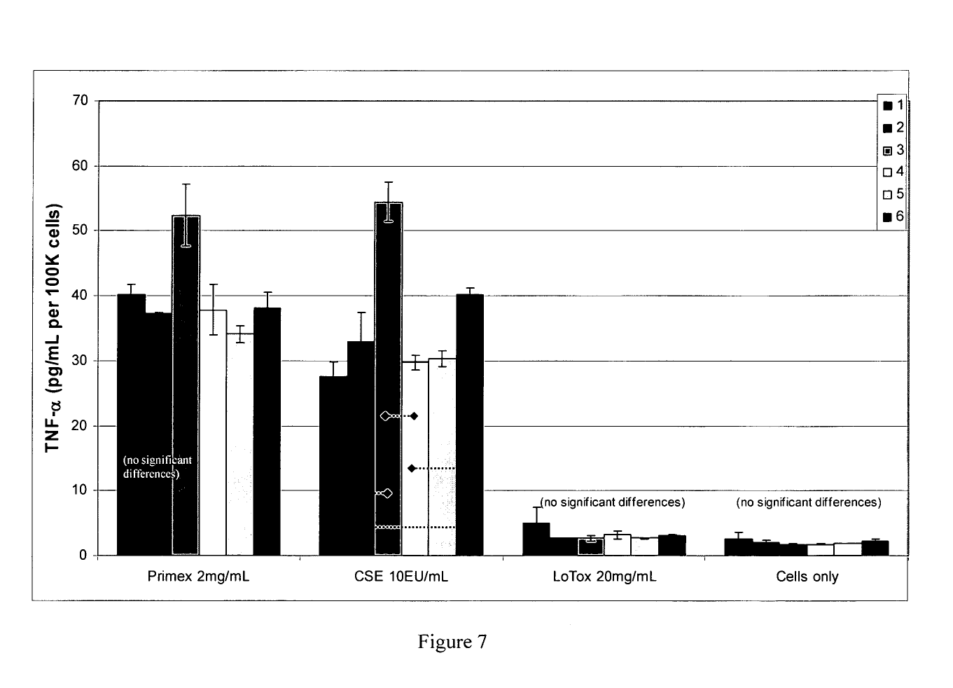

The LoTox chitosan was tested in a similar cell-based assay for its immune stimulating activity and compared to a variety of sub-clones of the cell population. All dendritic cells, including monocytes have some genetic variability in their response to stimuli. THP-1 human monocyte cell line (ATCC) was grown in a variety of conditions, passed and frozen a variety of times to produce different populations as designated 1-6 in FIG. 7. FIG. 7 shows the TNF-α response of 6 different cultures of THP-1 monocytes grown as explained in Example 5 and exposed to 2 mg/mL of food grade chitosan (Primex) and 20 mg/mL of ultra-pure chitosan (LoTox) as well as a cells only control and a control of 10 EU/mL of control standard endotoxin (CSE). A negative control with the subsets of THP-1 cells exposed to media is included as a control. The concentration of inflammatory cytokine TNF-α was measured by an ELISA technique at 1 hour after exposure to these materials. As expected, some variability in cellular response is seen in the subpopulations across all exposures of the food grade chitosan (Primex) and the endotoxin (CSE), as is observed in a random human population. Even with an order of magnitude less material, the food grade chitosan produced a large TNF-α response, comparable to 10 EU/mL, whereas the ultra-pure chitosan produced no significant response over cell control at 1 hour. Further, the cells do not have a statistically different response for the chitosan, although differences with p<0.05 are denoted as dashed lines with triangles between statistically significantly different measurements in the CSE measurements. No significant inflammatory response is observed for the medical grade, ultra-pure chitosan.

What is claimed is:

1. A process for making ultrapure, low-endotoxin chitosan comprising:
(a) utilizing sterile, pyrogen-free labware, reagents and materials in strict endotoxin-free, sterile environment;
(b) swelling chitosan having endotoxins, for up to 24 hours;
(c) dissolving ratios equivalent to 1 kg/25 L to 1.5 kg/25 L of said chitosan in 0.01M to 4.0 M of a hydroxide base, forming a chitosan base solution and continuously stirring said chitosan base solution
(d) heating said base solution between 60° C. and 100° C. for 45 minutes to 4 hours and continuously stirring said heated base solution;
(e) rinsing said chitosan solution with up to 10× volume of ultra-pure pyrogen-free water and removing endotoxin fragments and residual endotoxin;
(f) neutralizing said solution to a pH between 6.8 and 7.5;
(g) forming a ultra-pure low endotoxin chitosan slurry;
(h) transferring said slurry to a endotoxin-free closed system;
(i) removing excess water from said slurry and forming said ultra-pure, low-endotoxin chitosan having an endotoxin concentration between 100 EU/g and 20 EU/g;
(j) retaining molecular weight integrity of said chitosan at 30 kDa or greater and between 75% and 90% of said chitosan having endotoxins; and
(k) storing said chitosan in endotoxin-free storage containers in an endotoxin-free environment with sterile, endotoxin-free connects.

2. A process as recited in claim 1 wherein said chitosan having endotoxins is selected from a group consisting of food grade chitosan and commodity grade chitosan.

3. A process as recited in claim 2 wherein said base is NaOH.

4. A process as recited in claim 3 and further comprising processing said chitosan solution for 1 hour at 90° C. in 1.0 M NaOH.

5. A process as recited in claim 4 and further comprising removing said water by a process selected from the group consisting of filtration, aspiration or decanting.

6. A process as recited in claim 5, wherein said low endotoxin chitosan having an endotoxin concentration of 20 EU/g or less.

7. A process as recited in claim 6 and further comprising utilizing pyrogen-free water for swelling said chitosan.

8. A process as recited in claim 6 and further comprising utilizing an ultrapure 10% methanol solution.

9. An ultrapure low endotoxin chitosan product formed from a high endotoxin chitosan having a molecular weight of 30 kDa or greater, said low endotoxin chitosan having an endotoxin concentration between 100 EU/g and 20 EU/g and a molecular weight between 75% and 90% of said high endotoxin chitosan molecular weight.

10. A chitosan product as recited in claim 9, wherein said endotoxin concentration is 20 EU/g or less.

11. A chitosan product as recited in claim 10, wherein said chitosan is a paste, housed in an endotoxin-free, sterile storage environment.

12. A chitosan product as recited in claim 10, wherein said chitosan is available in an aqueous solution, housed in an endotoxin-free, sterile storage environment.

13. A process for making ultra-pure, low endotoxin chitosan comprising:
(a) utilizing sterile, pyrogen-free labware, materials and reagents;
(b) utilizing an endotoxin-free, sterile processing environment;
(c) processing starting materials so as to form chitin;
(d) deacetylizing and deproteinating said chitin with a strong base and forming said ultra-pure, low endotoxin chitosan having an endotoxin concentration between 100 EU/g and 20 EU/g; and
(e) packaging, handling and storing said low endotoxin chitosan in an endotoxin-free, sterile environment

14. A process as recited in claim 13 wherein said endotoxin concentration of said low endotoxin chitosan is 20 EU/g or less and said molecular weight of said chitosan is 30 kDa or greater.

15. A process for determining pyrogenicity of chitosan
(a) utilizing sterile and pyrogen-free materials, reagents and labware in a sterile, endotoxin-free environment;
(b) growing and priming human dendritic cells in standard media utilizing pyrogen-free serum;
(c) delivering between 2 μg/mL to 20 mg/mL of chitosan to equivalent wells of said dendritic cells;
(d) utilizing food grade chitosan as control and validating said cell positive response to said chitosan having endotoxins;
(e) bracketing said response with known pyrogens;
(f) periodically testing media from said wells for inflammatory and anti-inflammatory cytokine response;
(g) quantifying mass of said media utilizing ELISA plates of cytokine antibodies;
(h) retaining said wells so as to develop a pyrogen response; and
(i) detecting said pyrogen response in said media.

16. A process as recited in claim 15, wherein said chitosan concentration is 2 mg/mL or less.

17. A process as recited in claim 16, wherein said periodic testing occurs at 1 to 24 hours.

18. A process as recited in claim 17, wherein said period testing occurs at 1 hour for IL-6, IL-1β and TNF-α.

19. A process as recited in claim 18, further comprising utilizing cell lines for two to three months and priming said cells with a primer selected from the group consisting of calcitriol and interferon-γ.

20. A process as recited in claim 19 wherein said cells are primary cells.

21. A process as recited in claim 20, wherein said periodic testing includes testing for IL-6, IL-1β, TNF-α, IL-10, IL-4.

22. A process as recited 21 in claim wherein said chitosan comprises chitosan derivatives.
